(19) United States
(12) Patent Application Publication
(10) Pub. No.: US 2024/0016986 A1
(43) Pub. Date: Jan. 18, 2024

(54) METHOD FOR THAWING A BIOLOGICAL SAMPLE

(71) Applicant: LYO-VITA LTD., Ness Ziona (IL)

(72) Inventor: Amir ARAV, Tel Aviv (IL)

(73) Assignee: A.A. CASH TECHNOLOGY LTD., Tel Aviv (IL)

(21) Appl. No.: 18/038,528

(22) PCT Filed: Nov. 25, 2021
(86) PCT No.: PCT/IL2021/051407

§ 371 Date: May 24, 2023

Related U.S. Application Data
(60) Provisional application No. 63/118,667, filed on Nov. 26, 2020.

(51) Int. Cl. A61M 1/02; A01N 1/02

(52) U.S. Cl. CPC A61M 1/0281; A01N 1/0284; A01N 1/0289

(57) ABSTRACT

The present invention is directed to a method for thawing a frozen biological sample including subjecting the frozen biological sample to a temperature ranging from 30° C. to 80° C. and simultaneously applying a negative pressure to the frozen biological sample. Further provided is a biological sample thawed according to the method of the invention.

### CROSS-REFERENCE TO RELATED APPLICATIONS

This application claims the benefit of priority of U.S. Provisional Application No. 63/118,667, titled “METHOD FOR THAWING A BIOLOGICAL SAMPLE”, filed Nov. 26, 2020, the contents of which are incorporated herein by reference in their entirety.

### FIELD OF THE INVENTION

The present invention relates to rapid thawing of a frozen biological sample, such as comprising cells, for example red blood cells.

### BACKGROUND

Long term preservation of blood products for transfusion purposes has been of great scientific and practical interest over the past decade. In modern blood banking system, red blood cells (RBC) units are stored refrigerated (2-8° C.) in a liquid state up to a maximum duration of 42 days, depending on the preservative solution used. Degradation of the RBC under these storage conditions continues and has been demonstrated through alterations in cell content of adenosine three phosphate (ATP), 2,3-Diphosphoglycerate (DPG), and by alteration in RBC membrane deformability properties. These changes can be demonstrated as early as 2 weeks after collection, making the use of “old” units undesirable in emergency cases, such as severe bleeding.

Freezing, including cryopreservation allows for long term storage of RBC units. Only units collected from rare type donors are cryopreserved today, accounting for less than 1% of the collected blood units. There are 2 accepted cryopreservation methods; both employ the use of a cryoprotecting agent (CPA), such as glycerol. One method uses 20% (v/v) glycerol, and the unit is stored in liquid nitrogen (LN) tanks and the other uses 40% (v/v) glycerol and storage is done in −80° C. mechanical freezers.

However, the use of cryopreserved RBC units is extremely limited due to the cumbersome handling required for freezing, and even more so the complicated and time-consuming process required for thawing, which severely affects the survival and functionality of the thawed cells.

Further, a CPA, e.g., glycerol, DMSO, etc., is toxic to the designated subject, e.g., in need of transfusion, therefore a washing step must be performed upon thawing so as to remove the CPA. Additionally, the CPA is highly toxic to cells in ambient temperature, therefore, the thawing should provide a liquid thawed sample having lower temperature than the ambient temperature so as to efficiently remove the CPA while maintaining high cell survival rate. These elements are crucial in order to provide a subject with an adequate and effective transfusion. Contrary to these principals, current thawing and washing procedure is time consuming, taking between 1-2 hours. In particular, the thawing and CPA removal (e.g., termed deglycerolization in the case when glycerol is the CPA) render the use of frozen (e.g., cryopreserved) units unpractical under trauma scenarios, for example, in battlefield.

Therefore, there is still a great need for a method of rapid thawing of a frozen biological sample, e.g., comprising cells, wherein a liquid thawed sample is obtained, which is characterized by a temperature lower than ambient temperature, which will enable the efficient removal of CPA while maintaining increased cell survival rates, and subsequent transfusion.

### SUMMARY

In some embodiments, the present invention is directed to a rapid thawing of a biological sample comprising the heating of a frozen biological sample while simultaneously applying negative pressure so as to continuously vacuum a liquid thawed portion of the sample to a collecting compartment.

In some embodiments, the present invention is based, in part, on the surprising findings that by subjecting a frozen biological to a temperature of about 40° C. to 80° C. simultaneously to the application of negative pressure that is sufficient for continuously vacuuming a liquid thawed portion derived from the frozen biological sample to a collecting compartment, reduced the time of thawing. Further surprising is the fact that by simultaneously applying heat and negative pressure, over-heating of the sample during thawing was avoided, and a thawed sample having a temperature of 15° C. or lower was obtained. This result is in sharp contrast to the control, wherein negative pressure was not applied, and a thawed sample having a temperature of greater than 30° C. was obtained. To this end, a biological sample comprising cells and preserved by freezing, e.g., cryopreservation, requires the use of a cryoprotecting agent(s). These cryoprotecting agents, e.g., DMSO, glycerol, etc., are known to be highly toxic to cells in ambient temperatures, therefore, the herein disclosed findings are of major importance due to the fact that a thawed biological sample having a temperature of 15° C. or less was rapidly and efficiently obtained, while maintaining high survival of cells.

According to a first aspect, there is provided a method for thawing a frozen biological sample comprising subjecting the frozen biological sample to a temperature ranging from 30° C. to 80° C. and simultaneously applying a negative pressure to the frozen biological sample, wherein a level of the applied negative pressure is sufficient for continuously vacuuming a liquid thawed portion derived from the frozen biological sample to a collecting compartment, thereby, thawing the frozen biological sample.

According to another aspect, there is provided a biological sample thawed according to the herein disclosed method, for use in a transfusion of a subject in need thereof.

In some embodiments, the level of the applied negative pressure ranges from 0.01 mbar to 10 bar.

In some embodiments, at least 95% of the liquid thawed portion is transferred to the collecting compartment within 1 to 10 minutes.

In some embodiments, at least 99% of the liquid thawed portion is transferred to the collecting compartment within 1 to 10 minutes.

In some embodiments, the temperature of the liquid thawed portion ranges from 1° C. to 22° C.

In some embodiments, the temperature of the liquid thawed portion ranges from 1° C. to 15° C.

In some embodiments, the biological sample comprises: red blood cells, platelets, umbilical cord blood, sperm, a cell line, blood plasma, or whole blood.

In some embodiments, the thawing allows the survival of at least 40% of cells of the biological sample.

In some embodiments, the biological sample has a surface area to volume ratio ranging from 0.4 cm²/ml to 3 cm²/ml.

In some embodiments, the biological sample has a thickness of 1 mm to 12 mm.

In some embodiments, the subjecting comprises contacting the frozen biological sample with at least two heating elements.

In some embodiments, the frozen biological sample has a temperature of −2° C. to −196° C.

In some embodiments, the frozen biological sample is contained in an elastic bag.

In some embodiments, the collecting compartment is sterile.

In some embodiments, the collecting compartment is configured to withstand and/or provide a negative pressure ranging from 0.01 mbar to 1 bar.

In some embodiments, the collecting compartment is connected to a vacuum pump.

In some embodiments, transferred is by means of gravitational force.

In some embodiments, transferred is by means of pressure applied: by the collecting compartment, to the collecting compartment, or both.

In some embodiments, the method further comprises a step of transplanting the liquid thawed portion to a subject in need thereof.

In some embodiments, the subject is in need of a blood transfusion.

In some embodiments, the subject is afflicted with trauma.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes and image showing a 0.5 L frozen bag aspirate during thawing into a 1 L vacuum chamber.

FIG. 2 includes an image showing a front profile of a large surface bag of 0.5 L.

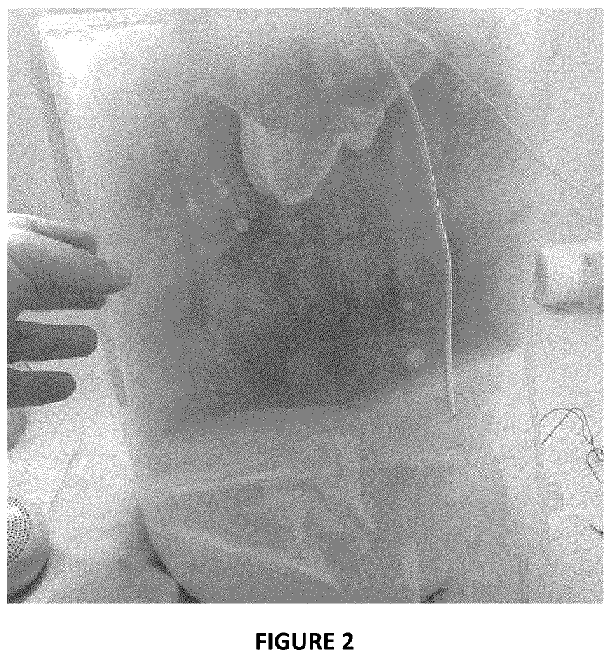

FIG. 3 includes an image showing a side profile of a large surface bag of 0.5 L (of FIG. 2) being less than 10 mm in width.

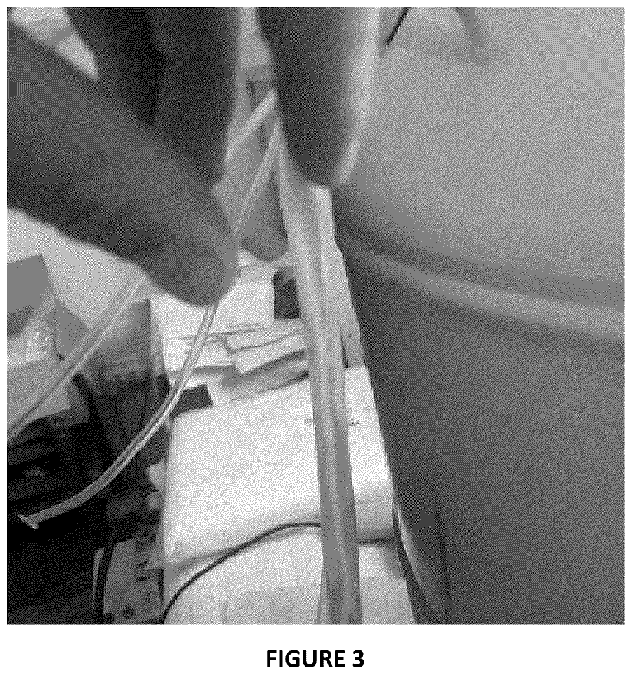

FIG. 4 includes an image showing a thermometer indicating that the temperature of a melted/thawed liquid 10 minutes after thawing was initiated was 13.4° C.

### DETAILED DESCRIPTION

According to some embodiments, there is provided a method for thawing a frozen biological sample comprising subjecting a frozen biological sample to a temperature ranging from 30° C. to 80° C. and simultaneously applying a negative pressure to the frozen biological sample.

In some embodiments, a level of the applied negative pressure is sufficient for continuously vacuuming a liquid thawed portion derived from the frozen biological sample to a collecting compartment, thereby, thawing the frozen biological sample.

In some embodiments, the method comprises subjecting the frozen biological sample to a temperature ranging from 30° C. to 80° C., 20° C. to 80° C., 35° C. to 80° C., 35° C. to 75° C., 40° C. to 80° C., 40° C. to 75° C., 45° C. to 80° C., 45° C. to 65° C., 50° C. to 85° C., ° C. to 70° C., or 45° C. to 55° C. Each possibility represents a separate embodiment of the invention.

In some embodiments, a level of negative pressure that is sufficient for continuously vacuuming a liquid thawed portion derived from the frozen biological sample to a collecting compartment ranges from: 0.01 mbar to 10 bar, 0.1 mbar to 500 mbar, 0.5 mbar to 100 mbar, 1 mbar to 10 bar, 3 mbar to 6 mbar, 50 mbar to 1,500 mbar, 1 bar to 50 bar, or 5 bar to 500 bar. Each possibility represents a separate embodiment of the invention. In some embodiments, level of negative pressure that is sufficient for continuously vacuuming a liquid thawed portion derived from the frozen biological sample to a collecting compartment is at least: 0.01 mbar, 0.1 mbar, 0.5 mbar, 1 mbar, 3 mbar, 6 mbar, 10 mbar, mbar, 100 mbar, 250 mbar, 500 mbar, 750 mbar, 1,000 mbar (e.g., 1 bar), 2 bar, 5 bar, bar, 50 bar, or 150 bar, or any value and range therebetween. Each possibility represents a separate embodiment of the invention.

In some embodiments, at least 95% of the liquid thawed portion is transferred to a collecting compartment within 1 to 3 minutes, 1 to 4 minutes, 1 to 5 minutes, 1 to 6 minutes, 1 to 7 minutes, 1 to 8 minutes, 1 to 9 minutes, 1 to 10 minutes, 2 to 7 minutes, 3 to 8 minutes, 4 to 9 minutes, or 5 to 10 minutes. Each possibility represents a separate embodiment of the invention.

In some embodiments, at least 99% of the liquid thawed portion is transferred to a collecting compartment within 1 to 3 minutes, 1 to 4 minutes, 1 to 5 minutes, 1 to 6 minutes, 1 to 7 minutes, 1 to 8 minutes, 1 to 9 minutes, 1 to 10 minutes, 2 to 7 minutes, 3 to 8 minutes, 4 to 9 minutes, or 5 to 10 minutes. Each possibility represents a separate embodiment of the invention.

In some embodiments, 100% of the liquid thawed portion is transferred to a collecting compartment within 1 to 3 minutes, 1 to 4 minutes, 1 to 5 minutes, 1 to 6 minutes, 1 to 7 minutes, 1 to 8 minutes, 1 to 9 minutes, 1 to 10 minutes, 2 to 7 minutes, 3 to 8 minutes, 4 to 9 minutes, or 5 to 10 minutes. Each possibility represents a separate embodiment of the invention.

In some embodiments, at least 50%, at least 60%, at least 70%, at least 80%, at least 90%, at least 95%, at least 97%, at least 99%, or 100% of the frozen biological sample, by weight, is thawed and transferred to a collecting compartment within 1 to 3 minutes, 1 to 4 minutes, 1 to 5 minutes, 1 to 6 minutes, 1 to 7 minutes, 1 to 8 minutes, 1 to 9 minutes, 1 to 10 minutes, 2 to 7 minutes, 3 to 8 minutes, 4 to 9 minutes, or 5 to 10 minutes. Each possibility represents a separate embodiment of the invention.

In some embodiments, the temperature of a liquid thawed portion ranges from 1° C. to 5° C., 1° C. to 10° C., 1° C. to 15° C., 1° C. to 22° C., 3° C. to 15° C., 5° C. to 10° C., 5° C. to ° C., or 4° C. to 16° C. Each possibility represents a separate embodiment of the invention.

In some embodiments, a biological sample is derived from a subject.

In some embodiments, a biological sample comprises red blood cells, platelets, umbilical cord blood, sperm, a cell line, blood plasma, whole blood, or any combination thereof.

In some embodiments, the thawing allows the survival of at least 30%, at least 40%, at least 45%, at least 50%, at least 60%, at least 65%, at least 70%, at least 75%, at least 80%, at least 85%, at least 90%, at least 95%, at least 97%, at least 99%, or 100%, of cells of the biological sample, or any value and range therebetween. Each possibility represents a separate embodiment of the invention.

In some embodiments, the biological sample has a surface area to volume ratio ranging from 0.3 cm²/ml to 3.5 cm²/ml, 0.4 cm²/ml to 3.0 cm²/ml, 0.5 cm²/ml to 2.9 cm²/ml, cm²/ml to 2.8 cm²/ml, 0.5 cm²/ml to 2.5 cm²/ml, 0.4 cm²/ml to 2.2 cm²/ml, 0.6 cm²/ml to 1.9 cm²/ml, 0.6 cm²/ml to 1.8 cm²/ml, 0.4 cm²/ml to 1.7 cm²/ml, or 0.5 cm²/ml to 2.0 cm²/ml. Each possibility represents a separate embodiment of the invention.

In some embodiments, the biological sample has a thickness of 1 mm to 6 mm, 1 mm to 7 mm, 1 mm to 8 mm, 1 mm to 9 mm, 1 mm to 10 mm, 1 mm to 11 mm, 1 mm to 12 mm, 3 mm to 12 mm, 4 mm to 11 mm, 4 mm to 9 mm, 2 mm to 9 mm, 3 mm to 8 mm, mm to 12 mm, or 3 mm to 10 mm. Each possibility represents a separate embodiment of the invention.

In some embodiments, subjecting comprises contacting the frozen biological sample with at least two, at least three, at least four, or at least five, heating elements, or any value and range therebetween. Each possibility represents a separate embodiment of the invention. In some embodiments, subjecting comprises submerging the frozen biological sample in a pre-heated liquid having a temperature ranging from 30° C. to 80° C., or any value and range therebetween such as disclosed herein. In some embodiments, subjecting comprises applying a fluid having a temperature ranging from 30° C. to 80° C., or any value and range therebetween such as disclosed herein to the frozen biological sample.

In some embodiments, the vacuuming results in a frozen surface of the frozen biological sample being in contact with the at least two heating elements. In some embodiments, the vacuuming results in a frozen surface of the frozen biological sample being in contact with the pre-heated liquid having a temperature ranging from 30° C. to 80° C., or any value and range therebetween. In some embodiments, the vacuuming results in a frozen surface of the frozen biological sample being in contact with the fluid having a temperature ranging from 30° C. to 80° C., or any value and range therebetween.

In some embodiments, the vacuuming results in the transferring of any liquid thawed portion derived from the frozen biological sample to the collecting compartment, such that a frozen surface of the frozen biological sample is in contact with the at least two heating elements.

In some embodiments, the vacuuming results in the transferring of any liquid thawed portion derived from the frozen biological sample to the collecting compartment, such that a frozen surface of the frozen biological sample is constantly in contact with the at least two heating elements.

In some embodiments, the vacuuming results in the transferring of any liquid thawed portion derived from the frozen biological sample to the collecting compartment, such that a frozen surface of the frozen biological sample is in contact with the pre-heated liquid having a temperature ranging from 30° C. to 80° C., or any value and range therebetween.

In some embodiments, the vacuuming results in the transferring of any liquid thawed portion derived from the frozen biological sample to the collecting compartment, such that a frozen surface of the frozen biological sample is constantly in contact with the pre-heated liquid having a temperature ranging from 30° C. to 80° C., or any value and range therebetween.

In some embodiments, the vacuuming results in the transferring of any liquid thawed portion derived from the frozen biological sample to the collecting compartment, such that a frozen surface of the frozen biological sample is in contact with the fluid having a temperature ranging from 30° C. to 80° C., or any value and range therebetween.

In some embodiments, the vacuuming results in the transferring of any liquid thawed portion derived from the frozen biological sample to the collecting compartment, such that a frozen surface of the frozen biological sample is constantly in contact with the fluid having a temperature ranging from 30° C. to 80° C., or any value and range therebetween.

As an exemplary non-limiting explanation, the herein disclosed method utilizes negative pressure to constantly transfer any liquid portion thawed from a frozen biological sample to a collecting compartment, wherein only a frozen portion yet to be thawed and transferred remains or is in contact with the heat providing elements, such as described herein, e.g., heating elements, pre-heated liquid, or fluid.

In some embodiments, constantly is until the frozen biological sample is fully or completely thawed.

The terms “completely” and “fully” are used herein interchangeably.

In some embodiments, a fluid comprises or consists of a liquid or a gas.

In some embodiments, the gas is an inert gas.

Types and applications of inert gas would be apparent to one of ordinary skill in the art.

In some embodiments, the frozen biological sample has a temperature of −2° C. to −196° C., −2° C. to −180° C., −2° C. to −170° C., −2° C. to −160° C., −2° C. to −140° C., −2° C. to −120° C., −2° C. to −100° C., −2° C. to −90° C., −2° C. to −80° C., −2° C. to −70° C., −2° C. to −60° C., −2° C. to −50° C., −2° C. to −40° C., −2° C. to −35° C., −2° C. to −30° C., −2° C. to −25° C., −2° C. to −20° C., −2° C. to −15° C., or −2° C. to −10° C. Each possibility represents a separate embodiment of the invention.

In some embodiments, the frozen biological sample is a cryopreserved biological sample. In some embodiments, the frozen biological sample comprises a cryoprotecting agent (CPA). In some embodiments, the frozen biological sample comprises at least one CPA. In some embodiments, the frozen biological sample comprises a plurality of CPAs.

As used herein, the term “plurality” refers to any integer equal to or greater than 2.

As used herein, the terms “cryoprotecting agent (CPA)” or “cryoprotectant” refer to any substance which is used to protect a biological cell or tissue from freezing damage. In some embodiments, a freezing damage results from the formation of ice crystals.

Types of CPAs suitable for cryopreservation of a biological sample, e.g., such as including cells as disclosed herein, are common and would be apparent to one of ordinary skill in the art. Non-limiting examples of such CPAs include, but are not limited to, DMSO, trehalose, glycerol, ethylene glycol, 2-Methyl-2,4-pentanediol (MPD), and propylene glycol, to name a few.

In some embodiments, the frozen biological sample is contained in a bag or a tube.

In some embodiments, the bag is an elastic bag.

In some embodiments, the collecting compartment is sterile. In some embodiments, the collecting tube is directly connected to the bag or tube comprising the biological sample, such as by one or more tubes. In some embodiments, the collecting tube is indirectly connected to the bag or tube comprising the biological sample, such as via a syringe.

In some embodiments, the collecting compartment is configured to withstand and/or provide a negative pressure ranging from: 0.01 mbar to 1 bar, 0.1 mbar to 500 mbar, mbar to 100 mbar, 1 mbar to 10 mbar, or 3 mbar to 6 mbar. Each possibility represents a separate embodiment of the invention.

In some embodiments, the collecting compartment has a sealing element.

In some embodiments, the collecting compartment has a sealing element positioned at the top of the collecting compartment.

In one embodiment, the collecting compartment has the shape or form of a bottle.

In some embodiments, the collecting compartment is connected to a pump. In some embodiments, the collecting compartment is connected to a vacuum pump.

In some embodiments, the potion of the thawed sample is transferred to the collecting compartment by means of gravity or gravitational force. In some embodiments, the potion of the thawed sample is transferred to the collecting compartment by means of an induced gravitational force.

In some embodiments, the potion of the thawed sample is transferred to the collecting compartment by means of pressure. In some embodiments, pressure comprises a negative pressure. In some embodiments, pressure comprises vacuum. In some embodiments, the pressure is applied by the collecting compartment (e.g., so as to transfer the portion of the thawed sample to the collecting compartment). In some embodiments, the pressure is applied to the collecting compartment (e.g., so as to transfer the portion of the thawed sample to the collecting compartment). In some embodiments, the pressure is applied to the collecting compartment and by the collecting compartment (e.g., so as to transfer the portion of the thawed sample to the collecting compartment).

In some embodiments, the pressure is applied: by the collecting compartment, to the collecting compartment, or both, such that a different level of pressure is present in the frozen sample and the collecting bag.

In some embodiments, the pressure is applied: by the collecting compartment, to the collecting compartment, or both, such that a pressure gradient is obtained or formed between the frozen sample and the collecting bag. In some embodiments, the pressure is greater in the frozen sample. In some embodiments, the pressure is lower in the collecting compartment.

In some embodiments, the potion of the thawed sample is transferred to the collecting compartment by means of both gravitational force and pressure application, each of which as described herein.

In some embodiments, the method further comprises a step of transplanting the liquid thawed portion to a subject in need thereof. In some embodiments, transplanting comprises transfusing.

In some embodiments, the subject is in need of a blood transfusion. In some embodiments, the subject suffers from: a loss of blood, reduced hematocrit, reduced blood plasma volume, thrombocytopenia, anemia, need for organ transplantation, cancer, organ failure, or any combination thereof.

In some embodiments, the subject is in need of an organ transplantation. In one embodiment, the subject is in need of liver transplantation.

In some embodiments, the subject is afflicted with trauma.

What types of injuries are classified as “trauma” would be apparent to one of ordinary skill in the art.

In some embodiments, trauma comprises a firearm injury.

According to some embodiments, there is provided a biological sample thawed according to the herein disclosed method. In some embodiments, the biological sample is a liquid biological sample derived from a frozen biological sample. In some embodiments, the liquid biological sample is derived from a frozen biological sample and thawed according to the herein disclosed method.

In some embodiments, the biological sample thawed according to the herein disclosed method is used in a transplantation or transfusion of a subject in need thereof.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference. Other general references are provided throughout this document.

### Example 1

### Rapid Thawing of a Frozen Sample

The inventors have evaluated the best way to warm/thaw 0.5 L of a frozen liquid.

The inventors have examined the following variables: the surface area to volume ratio, the warming convection heat transfer (hot air vs liquid; both at 50° C.), and application of negative pressure (e.g., vacuum) or lack thereof (control).

The inventors have used: (a) a conventional blood bag of 25 cm×15 cm comprising 0.5 L of liquid solution (surface area/volume ratio of 0.75 cm²/ml); and/or an Interface Multigrad Technology (IMT) bag. freezing bag with dimensions of 25 cm×28 cm frozen horizontally in a −30° C. freezer (surface area/volume ratio of 1.4 cm²/ml); and (b) A vacuum bottle (Virtis, USA) of 1 L with 1 torr vacuum.

Warming was performed by applying hot air with a temperature of 50° C. or submerging in a water bath preheated to 50° C.

The time to achieve complete melting (or thawing) and the temperature of the fully melted liquid were recorded at the end of the thawing process.

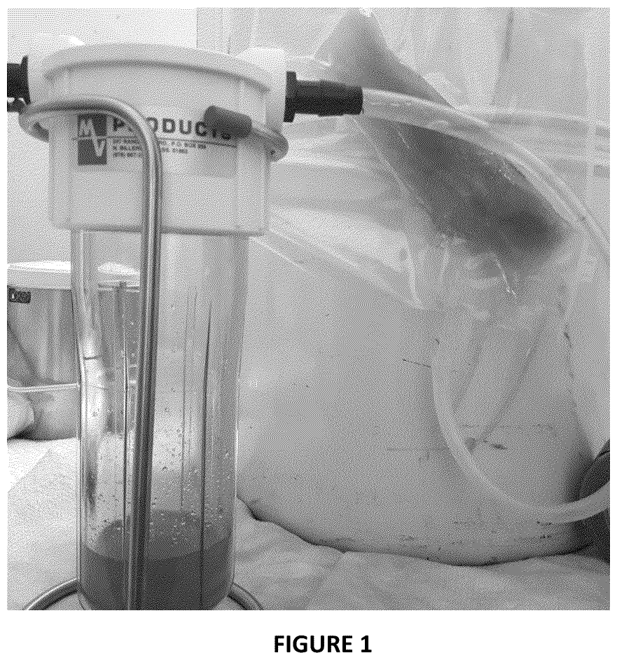

The inventors have started with a conventional blood bag having a surface area to volume ratio of 375 cm² (25 cm×15 cm)/500 ml. As shown in FIG. 1, the bag was connected vertically and warmed using hot air of 50° C. The bag was hanging in vertical position and the melted liquid was moved into the bottle by gravity. The duration of thawing was about 42 minutes long, and the fully melted solution was at a temperature of 23° C.

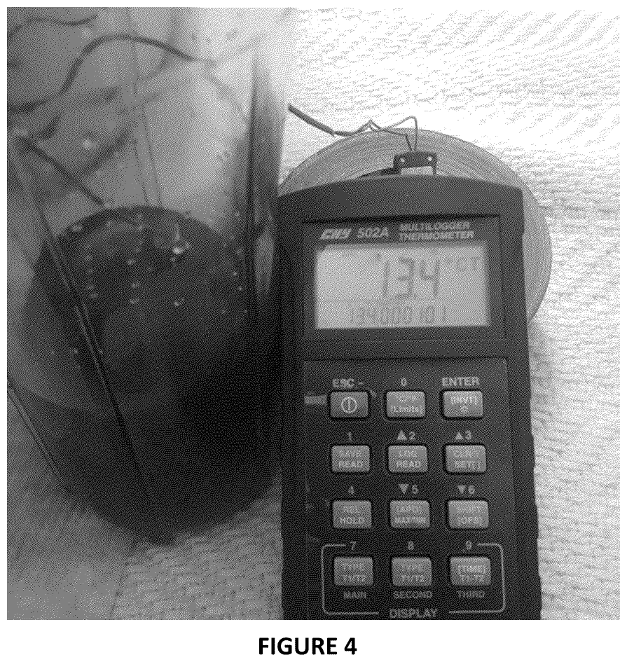

Further, the inventors have used an IMT blood bag having a surface area to volume ratio of 700 cm² (25 cm×28 cm)/500 ml. The warming was performed as with the conventional blood bag as described hereinabove. The bag was hanging in vertical position and the melted liquid was moved into the bottle by gravity. The duration of thawing of the IMT bag was only 10 minutes long, and the fully melted solution was at a temperature as low as 13.4° C. (FIG. 4).

Further, the inventors have examined the time to full thawing and temperature of the fully thawed solution, of an IMT blood bag warmed by submerging in a water bath preheated to 50° C. under vacuum conditions. The duration of complete thawing was dramatically reduced to 1:49 min, and the fully melted solution was at a temperature as low as 15.0° C.

In sharp contrast, a control frozen bag was examined as described hereinabove, with the exception of not including vacuum application or gravity force. The time to complete thawing of the control was longer than in the experimental group undergoing vacuum application and was recorded at 2 minutes and 40 seconds. Nonetheless, the fully melted solution over heated to a temperature of 34° C.

Further, the inventors found that transferring the thawed sample to the collecting compartment by means of gravity reduced the amount of pressure required so as to collect the thawed sample. Such combined transfer utilizing gravity and vacuum provided a fully thawed sample within 3:40 min and having a temperature of 15° C.

The inventors conclude that the thawing rate can be increased by increasing the size of the bag, e.g., to dimensions of 25 cm×28 cm. Increasing the bag's surface area by about 2-fold can reduce the thawing time significantly, either under negative pressure application, or by gravity force. Submerging the sample bag in a preheated water bath (e.g., of 50° C.) reduced the thawing time by more than 80%, compared to heating using hot air (1:49 minutes compared to 10 minutes, respectively). Such rapid thawing provided a fully melted solution with a temperature lower than the ambient temperature (e.g., 15° C.).

Further, the application of negative pressure reduced the time of thawing and prevented over-heating during thawing. The latter is of major importance as the temperature of the fully melted liquid did not exceed a temperature greater than 20° C. The ability to obtain a fully thawed biological sample derived from a frozen sample having a reduced temperature (lower than 20° C.) is crucial to avoid toxicity of cryoprotecting agents, e.g., DMSO, glycerol, etc., to cells. The use of such cryoprotecting agents is unavoidable and a necessity when preserving cells under freezing conditions, e.g., cryopreservation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1. A method for thawing a frozen biological sample comprising subjecting said frozen biological sample to a temperature ranging from 30° C. to 80° C. and simultaneously applying a negative pressure to said frozen biological sample, wherein a level of said applied negative pressure is sufficient for continuously vacuuming a liquid thawed portion derived from said frozen biological sample to a collecting compartment, thereby, thawing the frozen biological sample.

2. The method of claim 1, wherein said level of said applied negative pressure ranges from 0.01 mbar to 1 bar.

3. The method of claim 1, wherein at least 95% of said liquid thawed portion is transferred to said collecting compartment within 1 to 10 minutes.

4. The method of claim 1, wherein at least 99% of said liquid thawed portion is transferred to said collecting compartment within 1 to 10 minutes.

5. The method of claim 1, to wherein temperature of said liquid thawed portion ranges from 1° C. to 22° C.

6. The method of claim 1, wherein temperature of said liquid thawed portion ranges from 1° C. to 15° C.

7. The method of claim 1, wherein said biological sample comprises: red blood cells, platelets, umbilical cord blood, sperm, a cell line, blood plasma, or whole blood, and optionally wherein said thawing allows the survival of at least 40% of cells of said biological sample.

8. (canceled)

9. The method of claim 1, wherein said biological sample has a surface area to volume ratio ranging from 0.4 cm²/ml to 3 cm²/ml.

10. The method of claim 1, wherein said biological sample has a thickness of 1 mm to 12 mm.

11. The method of claim 1, wherein said subjecting comprises contacting said frozen biological sample with at least two heating elements.

12. The method of claim 1, wherein said frozen biological sample has a temperature of −2° C. to −196° C.

13. The method of claim 1, wherein said frozen biological sample is contained in an elastic bag.

14. The method of claim 1, wherein said collecting compartment is sterile.

15. The method of claim 1, wherein said collecting compartment is configured to withstand and/or provide a negative pressure ranging from 0.01 mbar to 1 bar.

16. The method of claim 1, wherein said collecting compartment is connected to a vacuum pump.

17. The method of claim 3, wherein said transferred is by means of gravitational force.

18. The method of claim 3, wherein said transferred is by means of pressure applied: by said collecting compartment, to said collecting compartment, or both.

19. The method of claim 1, further comprising a step of transplanting said liquid thawed portion to a subject in need thereof, and optionally wherein said subject is in need of a blood transfusion.

20. (canceled)

21. The method of claim 19, wherein said subject is afflicted with trauma.

22. A method for transfusing a subject in need thereof, the method comprising administering to said subject a therapeutically effective amount of a biological sample thawed according to the method of claim 1.
